(19) United States
(12) Patent Application Publication
(10) Pub. No.: US 2014/0154234 A1
(43) Pub. Date: Jun. 5, 2014

(54) METHOD FOR GENERATING THROMBIN

(71) Applicant: Biomet Biologics, LLC, Warsaw, IN (US)

(72) Inventors: Randel Dorian, San Diego, CA (US); Richard W. Storrs, Berkeley, CA (US); Michael D. Leach, Warsaw, IN (US)

(73) Assignee: Biomet Biologics, LLC, Warsaw, IN (US)

(21) Appl. No.: 14/063,648

(22) Filed: Oct. 25, 2013

Related U.S. Application Data
(63) Continuation of application No. 12/846,944, filed on Jul. 30, 2010, now abandoned.

(51) Int. Cl. C12N 9/74; A61K 38/48

(52) U.S. Cl. CPC C12N 9/6429; A61K 38/4833
USPC 424/94.64; 435/214

(57) ABSTRACT

Methods of generating thrombin and methods of applying a clotting tissue sealant to a site on a subject are provided. A blood component comprising platelets can be obtained from the subject. A hypotonic composition is contacted with a solid matrix to form a thrombin-containing liquid, where the hypotonic composition includes water, calcium, a blood component comprising platelets, and optionally a chelator. Calcium is present in the hypotonic composition in an amount greater than the amount of calcium that can be complexed by the chelator. Thrombin-containing liquid is then separated from the hypotonic composition and can be applied to the site on the subject to form a clot, for example, by combination with fibrinogen.

### INTRODUCTION

The present technology relates to methods for generating thrombin, thrombin compositions, and the use of thrombin as a clotting factor, such as an autologous clotting factor.

Whole blood, such as human whole blood, contains various proteins, cells, and other components. For example, whole blood includes a plasma fraction which can include platelets. Whole blood and plasma also include clotting factors, such as thrombin, that can form a clot to heal a lesion or other opening in tissue or skin.

Thrombin is a multifunctional serine protease that can activate various clotting factors and can activate platelets. Thrombin can be generated from prothrombin by enzymatic cleavage of two sites on prothrombin by activated Factor X (Xa). Factor Xa activity is enhanced by binding to activated Factor V (Va), termed the prothrombinase complex. Once formed, thrombin-mediated proteolytic digestion of fibrinogen into fibrin monomer starts a reaction cascade that can lead to clot formation, which is typically the first step in wound healing. Thrombin can also function as a chemo-attractant to cells involved in wound healing and the resulting fibrin network has several functions including acting as a scaffold for collagen-producing fibroblasts, increasing phagocytosis, promoting angiogenesis, and binding growth factors that can further support the healing process. Platelets are also activated from the nonbinding to the binding mode. As a procoagulant, thrombin plays an important role in the arrest of bleeding; i.e., physiological hemostasis.

Rate of clot formation can be dependent on the concentration of thrombin and fibrinogen. Because of its important function in clot formation, thrombin can be utilized as a tissue sealant or glue and can be used in conjunction with fibrinogen. Applications for wound sealants comprising thrombin are numerous and include uses in skin grafting, neurosurgery, cardiac surgery, thoracic surgery, vascular surgery, oncologic surgery, plastic surgery, ophthalmologic surgery, orthopedic surgery, trauma surgery, head and neck surgery, gynecologic and urologic surgery, gastrointestinal surgery, dental surgery, drug delivery, tissue engineering, and dental cavity hemostasis, among others.

### SUMMARY

The present technology includes methods and compositions that relate to generating thrombin, where the thrombin can be used as an autologous clotting factor, for example.

A method of generating thrombin comprises contacting a hypotonic composition with a solid matrix to form a thrombin-containing liquid. The hypotonic composition comprises water, calcium, a blood component comprising platelets, and optionally a chelator. The calcium is present in an amount greater than the amount of calcium that can be complexed by the chelator. The thrombin-containing liquid is separated from the hypotonic composition.

A method of applying a clotting tissue sealant to a site on a subject comprises obtaining a blood component comprising platelets. A hypotonic composition is contacted with a solid matrix to form a thrombin-containing liquid, where the hypotonic composition comprises water, calcium, the blood component comprising platelets, and optionally a chelator. The calcium is present in an amount greater than the amount of calcium that can be complexed by the chelator. The thrombin-containing liquid is separated from the hypotonic composition and applied to the site on the subject to form a clot.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic illustration of a representative method for generating thrombin according to an embodiment of the present technology;

FIG. 2 is a schematic illustration of a representative method for generating thrombin according to another embodiment of the present technology; and

FIG. 3 is a schematic illustration of a representative method for generating thrombin and for applying a clotting tissue sealant to a site on a subject according to another embodiment of the present technology.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

The present technology relates to methods of generating thrombin and compositions comprising thrombin generated using such methods. The present technology further provides methods of applying a clotting tissue sealant to a site on a subject. For example, the clotting tissue sealant can include thrombin produced according to the present methods that can be mixed with fibrinogen and applied to form a clot or fibrin glue. The thrombin converts the fibrinogen to fibrin which forms a clot or wound sealant. The clot or sealant can be applied to an incision site following surgery or to seal a wound, for example. The clot or sealant can also facilitate healing at the site.

Methods of generating thrombin include a step of contacting a hypotonic composition with a solid matrix to form a thrombin-containing liquid. The hypotonic composition includes water, calcium, a blood component including platelets, and optionally a chelator. For example, the blood component may be whole blood or a blood fraction such as platelet rich plasma. The calcium is present in the hypotonic composition in an amount greater than the amount of calcium that can be complexed by the chelator, if the optional chelator is present. For example, where the blood component includes a chelator, such as citrate, to prevent coagulation, enough calcium is added so that the citrate cannot form a chelate complex with all of the calcium. The thrombin-containing liquid is separated from the hypotonic composition, for example, where the liquid is withdrawn from the solid matrix.

Methods of applying a clotting tissue sealant to a site on a subject include obtaining a blood component comprising platelets, such as whole blood or platelet rich plasma. A hypotonic composition including water, calcium, the blood component comprising platelets, and optionally a chelator is then contacted with a solid matrix to form a thrombin-containing liquid. The calcium in the hypotonic composition is present in an amount greater than the amount of calcium that can be complexed by the chelator if the optional chelator is present. The thrombin-containing liquid is separated from the hypotonic composition and applied to the site on the subject to form a clot. In some methods, the blood component comprising platelets is obtained from the subject. And in some methods, the thrombin-containing liquid is combined with fibrinogen and applied to a site on the subject.

In some embodiments, the present methods can be used to create autologous thrombin for use as a clotting factor, where the thrombin originates from a blood component comprising platelets, such as whole blood or platelet rich plasma (PRP). The thrombin can be used to clot various blood components at a point of care, where the blood component to be clotted can include purified fibrinogen, whole blood, platelet rich plasma (PRP), platelet concentrate, platelet poor plasma (PPP) or concentrates thereof. The blood component may contain a chelator, such as citrate, to chelate calcium ions and prevent coagulation. Excess calcium is therefore added to overwhelm the chelator and provide free calcium ions for the blood-clotting cascade.

For various treatments and reasons, a concentration of clotting factors, such as thrombin, can be provided or applied at a particular location on or within a subject. For example, during a surgical procedure, such as an orthopedic surgical procedure, a concentration of clotting factors can be provided at an incision site, an implantation site, or a repair site. The clotting factors can assist in healing the incision in tissue by sealing the site and can assist the body in healing thereafter.

Thrombin and other clotting components may be generated from autologous, homologous, or heterologous sources. For example, bovine thrombin can be prepared for use as a clotting factor when performing a procedure on a human. Thrombin may also be obtained from a homologous source, such as a compatible human donor. However, it is often desirable to use methods and compositions that employ autologous thrombin and clotting factors to assist in reducing the possibility of infection, immune reaction, or other side effects from using a non-autologous source.

The calcium may comprise a calcium salt, such as calcium chloride, calcium carbonate, calcium sulfate, and combinations thereof. The calcium may be provided in a solid form, such as salt crystals which may be premeasured to provide a particular concentration based on a particular container volume. Or, the calcium may be provided as a concentrated solution that is diluted by the water and blood component to a final concentration that is greater than the amount of calcium that can be complexed by any chelator present in the blood component. In some embodiments, the water component of the hypotonic composition contains the calcium. This can simplify the formation of the hypotonic composition, for example, so that water comprising calcium and a blood component can be added to a container having the solid matrix already therein. Alternatively, the container may already hold the water comprising the calcium and the solid matrix so that only the blood component needs to be added to form the hypotonic solution. In some cases, a suspension of the solid matrix in hypotonic solution can be added to a container containing the blood component.

The hypotonic composition formed by combining water, calcium, the blood component, and the solid matrix is hypotonic in that the water dilutes the blood component and other portions of the composition so that the composition formed is hypotonic with respect to cells and platelets in the blood component. A hypotonic solution contains a lower concentration of solute(s) outside of the cells' membranes than the solution inside the cells' membranes, causing water to be drawn from outside and into the cells by osmosis. As water continues to enter into the cells, it causes the cells to swell due to osmotic pressure. In some embodiments, about two parts water is combined with about one part blood component. The concentration of platelets in the hypotonic solution may be less than about half of the concentration of platelets in whole blood and may be less than about one-third of the concentration of platelets in whole blood following addition of the water.

In some cases, the hypotonic composition contains more platelets than found in whole blood. This may be accomplished using a blood component such as platelet rich plasma, concentrated platelet rich plasma, or platelet concentrate. Dilution due to the water in the hypotonic composition, therefore, may make the composition hypotonic causing the platelets to swell and/or lyse; however, the amount of platelets present in the hypotonic composition may be equal to or greater than found in whole blood of the same volume.

Where the water component is water or water containing calcium, dilution of the blood component effectively unbalances the osmotic pressure in the cells. The water may comprise less sodium than isotonic saline and may include no sodium. In some cases, the water may comprise only water, such as distilled and/or deionized water, or may comprise only water and a calcium salt. Combining the water, calcium, a blood component comprising platelets, and the solid matrix may cause at least a portion of the platelets in the resulting hypotonic composition to lyse, where the cell membranes of a portion of the cells and/or platelets rupture, burst, or evert. In some embodiments, the hypotonic composition causes most or all of the platelets in the blood component to lyse.

The blood component comprising platelets can include whole blood, PRP, concentrated PRP, and/or platelet concentrate. In some cases, it may be possible to use or include PPP or concentrated PPP, which may not be completely devoid of platelets.

The blood component may include a chelator, such as citrate. For example, Acid Citrate Dextrose (ACD) is a solution of citric acid, sodium citrate, and dextrose in water that can be used as an anticoagulant to preserve blood. In some embodiments, the blood component may include ACD-A, which includes per 1000 mL: total citrate (as citric acid, anhydrous (C₆H₈O₇)) about 20.59 g to 22.75 g, dextrose (C₆H₁₂O₆*H₂O) about 23.28 g to 25.73 g, and sodium (Na) about 4.90 g to 5.42 g. In some embodiments, ACD-B is used, which includes per 1000 mL: total citrate (as citric acid, anhydrous (C₆H₈O₇)) about 12.37 g to 13.67 g, dextrose (C₆H₁₂O₆*H₂O) about 13.96 g to 15.44 g, and sodium (Na) about 2.94 g to 3.25 g. In some embodiments, the present methods include obtaining the blood component comprising platelets and adding a chelator to the blood component to prevent coagulation prior to forming the hypotonic composition. For example, platelet rich plasma derived from blood anticoagulated with 1/7 volume of ACD-A can be used with addition of a volume of 290 millimolar CaCl₂ equal to 1/10 the volume of anticoagulated plasma and a volume of water equal to twice the volume of anticoagulated plasma in the presence of solid matrix to generate thrombin.

The solid matrix can include various materials that provide a solid surface area to contact cells in the hypotonic composition. The solid matrix may be a continuous material or may be discontinuous and comprise a plurality of separate particles. The solid matrix may comprise geometric forms having various cross-sectional shapes, such as spherical, oval, or polygonal, among others. The solid matrix can also comprise a continuous porous network, similar to a sponge, or can include a plurality of individual porous particles. In some embodiments, the solid matrix includes particles having a large aspect ratio, for example, where the particles are needle-like in shape. The solid matrix may also be formed as long fibers or in some embodiments can comprise the internal walls of the container.

Where the solid matrix is a continuous material, such as a porous sponge-like material, the solid matrix can be used in an amount sufficient to soak up or include substantially the entire liquid portion (e.g., the water, calcium, and blood component comprising platelets) of the hypotonic composition within the pores or interstices of the solid matrix. Where the solid matrix is a discontinuous material, such as a plurality of particles, the solid matrix can be combined with the other components of the hypotonic composition to form a slurry. The slurry can vary in consistency from paste-like, having a high-solids fraction, to a readily flowable slurry having a low-solids fraction. In some embodiments, the solid matrix can include one or more of a collagen sponge, collagen beads, or a container wall coating of collagen.

The solid matrix provides a large surface area to contact the cells. In some cases, the solid matrix material can be treated to increase its surface area, for example, by chemically etching or eroding the surface of the solid matrix. The solid matrix may also provide a larger surface area by being porous. Various polymers, metals, ceramics, and glasses can be used as the solid matrix. These include, for example, a continuous solid matrix of glass or a plurality of glass particles, glass wool, a continuous solid matrix of titanium or a plurality of titanium beads or titanium powder, and combinations thereof. A continuous solid matrix of titanium can include a block or other three dimensional shape formed of porous titanium or titanium alloys with an open cell structure. The solid matrix may include various beads of various sizes including substantially spherical beads. Beads include polystyrene beads, polyacrylamide beads, glass beads, titanium beads, or any other appropriate beads. Beads may be any size appropriate for the container and the amount of hypotonic composition being formed. In some cases, bead sizes can range from about 0.001 millimeters to about 3 millimeters in diameter.

Surface contact with the solid matrix can activate various parts of the blood component, such as the platelets, and the solid matrix can, in some cases, assist in the separation and concentration of the clotting component including thrombin. For example, in the case of a porous solid matrix, a liquid portion of the hypotonic composition can enter the pores and remain therein. Platelets in the composition may contact this additional surface area. In some embodiments, the pores are too small for the platelets to enter, but components of the hypotonic composition that are smaller than the platelets can enter the pores. Liquid can be removed from the solid matrix and pores of the solid matrix by centrifuging, for example. In some embodiments, the solid matrix may include a hygroscopic material, such as desiccating polyacrylamide beads, that absorbs a liquid portion of the hypotonic composition, thereby concentrating materials outside the solid matrix that are not absorbed into the hygroscopic material.

Once the water, calcium, blood component comprising platelets, and solid matrix are combined to form a hypotonic composition, the thrombin-containing liquid is separated from the hypotonic composition. In some embodiments, the liquid is separated from the hypotonic composition shortly after the water, calcium, blood component comprising platelets, and solid matrix are combined. For example, at least about one minute may pass between contacting the hypotonic composition with the solid matrix and separating the thrombin-containing liquid from the hypotonic composition. In some cases, the hypotonic composition is left for several minutes, for about a half-hour, for about one hour, or for several hours before the thrombin-containing liquid is separated.

In some embodiments, the hypotonic composition is agitated prior to the separating step. The agitation may be accomplished by inverting, shaking, rocking, stifling, or vortexing the hypotonic composition. Agitation may increase contact of platelets with the solid matrix and at least a portion of the platelets may lyse due to the agitation. In some cases, a majority or substantially all of the platelets are lysed due to the agitation. Agitation may be performed once, repeated multiple times, repeated periodically, or may be continuous so that the thrombin-containing liquid is separated from the hypotonic composition. A combination of the hypotonic state of the cells in the hypotonic composition combined with agitation may cause a portion of the cells to lyse in some embodiments. The combination of the hypotonic state and the agitation may also cause the majority of the cells to lyse or substantially all of the cells to lyse.

Separating the thrombin-containing liquid from the hypotonic composition can be performed in various ways. For example, the thrombin-containing liquid may be removed from the hypotonic composition using a syringe, by filtering the hypotonic composition, by centrifuging the hypotonic composition, or by using methods suitable for separating a liquid from the solid matrix. These separation techniques may be combined; for example, where the liquid is removed with a syringe, the remaining hypotonic composition can be subjected to centrifugation, and any liquid that sediments may also be removed with the syringe. In some embodiments, the thrombin-containing liquid can pushed out from the composition using pressure or drawn out using vacuum.

In some embodiments, the container in which the hypotonic composition is assembled can be configured to aid in separating the subsequent thrombin-containing liquid portion from the hypotonic composition. For example, the container may include a mesh screen or glass frit on one side, on the bottom, or on the container lid. The container can then be centrifuged where the liquid passes through the mesh or frit and the solid matrix and other materials, such as platelets, are retained. In some cases, only the solid matrix is retained and substantially all of the other materials pass through the mesh or frit. In this manner, the liquid can be centrifuged and collected into a fresh container, for example.

The output thrombin-containing liquid that results from the present methods may be further treated or processed in various ways. Improved stability of the thrombin-containing liquid may be attainable by adding citrate or citric acid to chelate the calcium or adding inhibitors of degradative or inhibitory processes (e.g., an alcohol, such as glycerol or other polyol), or reducing temperature (e.g., to 0° C.) after activation. For example, the thrombin can be preserved for later use by adding glycerin to the thrombin-containing liquid, by adding a chelator (e.g., citrate) to complex calcium, the pH of the liquid can be adjusted to an acidic pH (i.e., below 7), and/or temperature can be reduced (e.g., to 0° C.). Such treatments can help preserve the thrombin and keep the liquid from continuing to clot or coagulate.

The thrombin-containing liquid may also be used as a wound sealant or glue to facilitate the closure of a surgical incision, for example. The thrombin-containing liquid can be used “as is” or can be combined with other clotting factors, blood components, or blood products. For example, the thrombin-containing liquid can be admixed with fibrinogen and applied to a wound, lesion, or incision as a fibrin glue. Thrombin can convert the fibrinogen into fibrin, for example in about 5 seconds or less to about 60 seconds or more, which then forms a fibrin scaffold that can seal the application site and can promote healing at the site.

In some embodiments, the thrombin-containing liquid is applied to the same subject from which the blood component comprising platelets was derived; i.e., the thrombin is autologous to the subject. Accordingly, autologous thrombin can be combined with autologous fibrinogen to form an autologous wound sealant for a subject. The present methods therefore afford preparation of an autologous clotting composition that can be prepared and used while a subject is undergoing a surgical procedure or to treat a subject presenting a lesion or trauma.

The present methods provide an unexpected increase in thrombin generation. The increase in thrombin activity resulting from dilution of the blood component comprising platelets (e.g., whole blood and/or PRP) with the water is in fact counterintuitive. By diluting the precursor prothrombin, one would expect on first principles to see a diminution in the generated thrombin concentration in proportion to the dilution factor. Even with a thorough understanding of complex hemostasis mechanisms, one would not readily anticipate the observed result of the present technology. In particular, the present methods can generate more thrombin than similar methods performed without combining water with the blood component.

Calcium-dependent conversion of prothrombin to thrombin in blood or plasma can be frustrated by factors and processes which degrade generated thrombin and also prevent further conversion via feedback inhibition. It is not only prothrombin activation that is calcium-dependent, as many other hemostatic interactions also require calcium, including some of the inhibitory and degradative processes; e.g., autodigestion of thrombin itself. The present methods use dilution of a blood component including platelets (e.g., citrated blood) with water including sufficient calcium to overwhelm any chelator (e.g., citrate) in the presence of solid matrix to activate prothrombin.

Without wishing to be bound by theory, there are a number of plausible explanations for the demonstrated efficiency of prothrombin activation in the blood component diluted with the water. It is likely that a combination of effects may be relevant. Rapid disruption of the platelets in blood or PRP, for example, by hypotonic shock may lead to release of soluble platelet-associated activation factors and simultaneous exposition of the inner surface of the everted platelets, which serves as substrate for some prothrombin-activating processes. Thrombin activation occurs by a number of pathways and mechanisms, the most efficient involving formation of a tenase complex associated with the everted platelet membrane. Reaction kinetics of this solid-phase complex are not affected by dilution of the aqueous suspending medium, but the dilution of soluble counterproductive (both inhibitory and catabolic) factors could reduce their effectiveness.

The complex interaction of factors and processes which drive the equilibrium also may be skewed by dilution effects even in the absence of a solid-phase reaction complex because of differential effects on the individual interplaying components. Because certain of the processes in clotting involve cascading reactions, the effects of dilution are likely to be non-linear.

Finally, there are likely to be allosteric effects of sodium on the thrombin and the various other proteins involved in the clotting cascade. It is interesting to note that the same dramatic prothrombin activation afforded by the present methods is not observed using isotonic saline as a plasma diluent. This is consistent with allostery being a significant factor; for example, the sodium ions may interfere with effects of calcium ions. But, the difference could alternatively be attributable to the effect of hypotonic shock on platelet integrity.

Adding high concentrations of sodium chloride to plasma abolishes thrombin activation, also consistent with the notion of allosteric effects of salts playing a role, but also rather likely attributable to simple salting-out effects. An observation which may lend support to the notion that dilution of inhibitory factors might play a significant role is that clot time after addition to concentrated PRP of thrombin generated through activation of prothrombin is considerably slower than after addition to PRP. Regardless of which of these several effects may in fact be playing out in diluting whole blood or PRP with the water, the observed effect of increased thrombin generation could not be foreseen and is unexpected. One does not normally expect an increased rate or generation of a material when the reaction medium is diluted.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that certain specific details need not be employed, that example embodiments may be embodied in many different forms, and that neither should be construed to limit the scope of the disclosure. In some example embodiments, well-known processes, well-known device structures, and well-known technologies are not described in detail.

### EXAMPLES

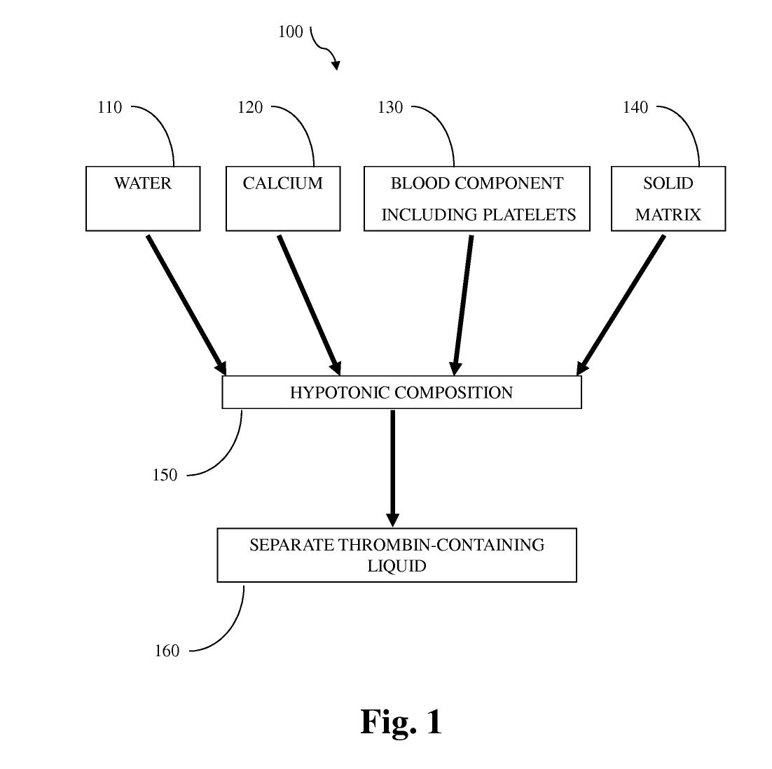

Referring now to FIG. 1, an embodiment of a method for generating thrombin 100 is shown. The method 100 includes combining water 110, calcium 120, a blood component comprising platelets 130, and a solid matrix 140 to form a hypotonic composition 150. The water 110 can be water only, such as sterile distilled or deionized water, a buffered aqueous solution, and/or can include various salts or preservatives. However, any solute present in the water 110 is limited so that following combination of the water 110, calcium 120, blood component 130, and the solid matrix 140, the resulting hypotonic solution 150 causes the cells in the blood component 130 to swell; i.e., the composition is hypotonic. For example, combination of sterile, distilled and deionized water with the other components in the hypotonic solution 150 will cause the platelets and any other cells in the blood component to swell and some may even lyse. Depending on the amount of water 110 added, the presence of any solute(s) in the water, and the amount of calcium added, water will be drawn from the solution into the cells by osmosis. If enough water molecules continue to diffuse into the cells, the cells can swell to the point that a portion of the cells lyse, or in some cases, even the majority of the cells or substantially all of the cells may lyse.

The calcium 120 can be added as a solid calcium salt, such as CaCl₂, or may be added as a predissolved calcium solution. The calcium 120 is added in an amount greater than the amount of calcium that can be complexed by a chelator present in the blood component comprising platelets 130. For example, the blood component can be whole blood and/or PRP to which a known volume of Acid Citrate Dextrose Formula A (ACD-A) was added. Accordingly, the amount of calcium added is based on the known amount of citrate in the ACD-A solution so that there is enough free calcium in the hypotonic composition 150 to participate in the clotting cascade. In some cases, calcium is added to a final concentration of about 3 to 30 millimolar.

The method 100 also includes separating a thrombin-containing liquid 160 from the hypotonic composition 150. Separation can be achieved using various means known in the art to separate liquids from the solid materials in the hypotonic composition 150. The thrombin in the liquid is soluble and the liquid can be aspirated from the hypotonic composition 150 using vacuum or a syringe, for example. In some embodiments, the entire liquid portion of the hypotonic composition 150 is separated in 160 to produce the thrombin-containing liquid. The liquid may therefore include all or most of the components added to form the hypotonic composition 150 except for the solid matrix 140. For example, the liquid separated in 160 may include cells and/or lysed cells, the calcium, along with the generated thrombin. In other embodiments, a liquid portion of the hypotonic composition 150 can be separated in 160 to exclude the solid matrix 140 as well as cells and/or lysed cells, for example, by filtering the hypotonic composition 150 with a filter having a desired size cut off.

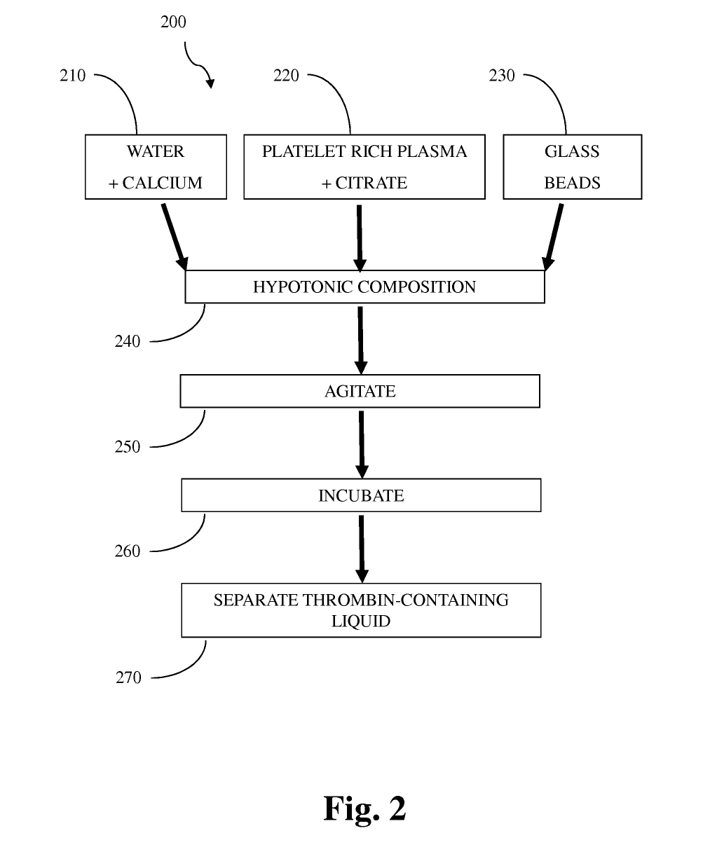

Referring now to FIG. 2, another embodiment of a method for generating thrombin 200 is shown. The method 200 includes combining two parts of sterile water containing calcium 210, one part of platelet rich plasma including citrate 220, and glass beads 230 in an amount to make a slurry and form a hypotonic composition 240. The amount of calcium in the sterile water 210 is enough to overwhelm the citrate in the platelet rich plasma 220. The hypotonic composition 240 is agitated, as shown at 250, by shaking vigorously so that the slurry of water including calcium 210, the platelet rich plasma including citrate 220, and the glass beads 230 are thoroughly mixed. Agitation 250 may also cause a portion of the platelets in the hypotonic composition 240 to lyse. After the agitation step 250, the composition is incubated as shown at 260 for about five minutes as the clotting cascade generates thrombin from prothrombin. As shown at 270, the thrombin-containing liquid is separated from the hypotonic composition 240, for example, by extracting the full volume of liquid using a syringe. The thrombin-containing liquid 270 can then be combined with fibrinogen and used as a wound sealant or can be preserved for later use.

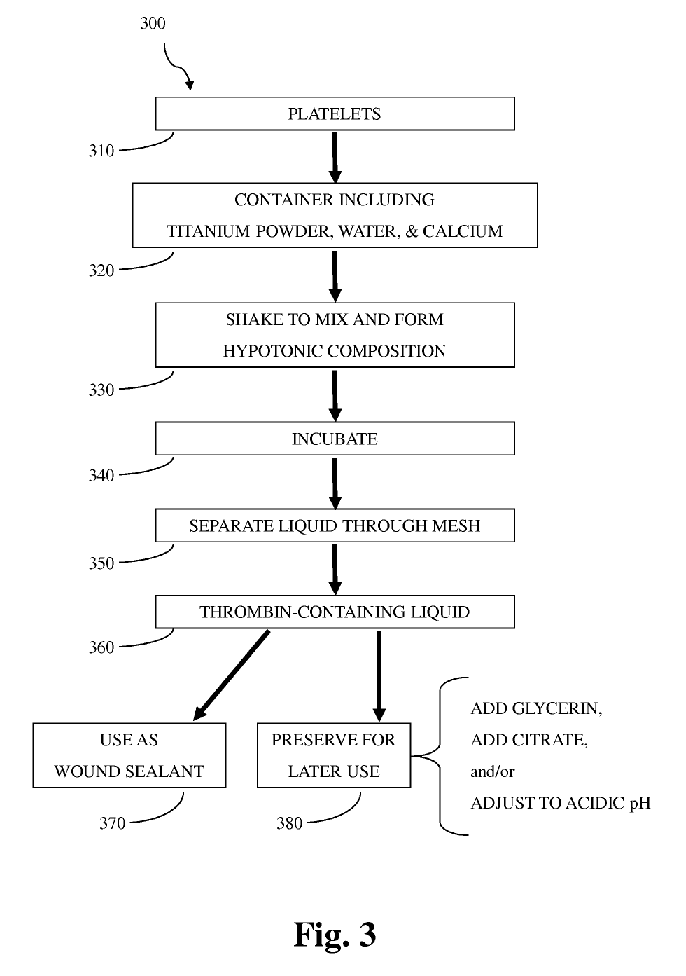

Referring now to FIG. 3, an embodiment of a method for generating thrombin and a method of applying a clotting tissue sealant to a site on a subject 300 is shown. The method 300 includes adding platelets 310 to a sterilized container including a slurry of titanium powder, water, and calcium 320. The calcium can be a calcium salt, such as calcium chloride, dissolved in the water. The platelets can be from a blood component, such as whole blood and/or platelet rich plasma, and can be anticoagulated by the addition of a citrate solution, for example. A hypotonic solution is formed by shaking the container to mix the platelets, titanium powder, water, and calcium, as shown at 330. The hypotonic composition is allowed to incubate 340 for a period of time. During the incubation 340, the container is optionally shaken one or more additional times or may be shaken throughout the incubation 340. The liquid portion of the hypotonic composition is separated 350 to provide a thrombin-containing liquid 360. For example, part of the container, such as the lid, can include or be replaced with a mesh filter able to retain the titanium powder but allow the thrombin-containing liquid to pass through when the container is centrifuged. For example, where the lid includes the mesh filter, the container can be inverted into a conical centrifuge tube, spun, and the liquid collected in the conical centrifuge tube leaving the titanium powder in the original container. Alternatively, the container may be centrifuged to pellet the titanium powder and the supernatant decanted or withdrawn for use as the thrombin-containing liquid 360.

The thrombin-containing liquid 360 can then be used as a wound sealant 370, for example, by combination with fibrinogen. Or, the thrombin-containing liquid 360 can be preserved for later use 380 by one or more of adding an alcohol (e.g., glycerin or other polyol), adding citrate, and adjusting the liquid to acidic pH.

The embodiments and the examples described herein are exemplary and not intended to be limiting in describing the full scope of apparatus, compositions, systems, and methods of the present technology. Equivalent changes, modifications and variations of some embodiments, materials, compositions and methods can be made within the scope of the present technology, with substantially similar results.

### Non-limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “desire” or “desirable” refer to embodiments of the technology that afford certain benefits, under certain circumstances. However, other embodiments may also be desirable, under the same or other circumstances. Furthermore, the recitation of one or more desired embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on”, “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

What is claimed is:

1. A method of generating thrombin comprising:
contacting a hypotonic composition with a solid matrix to form a thrombin-containing liquid, the hypotonic composition comprising water, calcium, a blood component comprising platelets, and optionally a chelator, wherein the calcium is present in an amount greater than the amount of calcium that can be complexed by the chelator;
separating the thrombin-containing liquid from the hypotonic composition.

2. The method of claim 1, wherein the water contains the calcium.

3. The method of claim 1, wherein the concentration of platelets in the hypotonic solution is less than about half of the concentration of platelets in whole blood.

4. The method of claim 1, wherein the concentration of platelets in the hypotonic solution is less than about one-third of the concentration of platelets in whole blood.

5. The method of claim 1, wherein the blood component comprises the chelator.

6. The method of claim 1, further comprising obtaining the blood component comprising platelets and adding a chelator to the blood component to prevent coagulation prior to the contacting step.

7. The method of claim 1, wherein the solid matrix comprises glass, titanium, or combinations thereof.

8. The method of claim 1, wherein at least about one minute passes between the contacting step and the separating step.

9. The method of claim 1, wherein the hypotonic composition and solid matrix are agitated prior to the separating step.

10. The method of claim 1, wherein the separating comprises removing the thrombin-containing liquid from the solid matrix using a syringe, by filtering, or by centrifuging.

11. The method of claim 1, further comprising at least one of: adding an alcohol to the thrombin-containing liquid; adding a chelator to complex calcium in the thrombin-containing liquid; adjusting the pH of the thrombin-containing liquid to below 7; and reducing the temperature of the thrombin-containing liquid.

12. A method of applying a clotting tissue sealant to a site on a subject comprising:
obtaining a blood component comprising platelets;
contacting a hypotonic composition with a solid matrix to form a thrombin-containing liquid, the hypotonic composition comprising water, calcium, the blood component comprising platelets, and optionally a chelator, wherein the calcium is present in an amount greater than the amount of calcium that can be complexed by the chelator;
separating the thrombin-containing liquid from the hypotonic composition; and
applying the thrombin-containing liquid to the site on the subject to form a clot.

13. The method of claim 12, wherein the water contains the calcium.

14. The method of claim 12, wherein the concentration of platelets in the hypotonic solution is less than about half of the concentration of platelets in whole blood.

15. The method of claim 12, wherein the concentration of platelets in the hypotonic solution is less than about one-third of the concentration of platelets in whole blood.

16. The method of claim 12, wherein the blood component comprises the chelator.

17. The method of claim 12, further comprising adding a chelator to the blood component to prevent coagulation prior to the contacting step.

18. The method of claim 12, wherein the solid matrix comprises glass, titanium, or combinations thereof.

19. The method of claim 12, wherein at least about one minute passes between the contacting step and the separating step.

20. The method of claim 12, wherein the hypotonic composition and solid matrix are agitated prior to the separating step.

21. The method of claim 12, wherein the separating comprises removing the thrombin-containing liquid from the solid matrix using a syringe, by filtering, or by centrifuging.

22. The method of claim 12, further comprising at least one of: adding an alcohol to the thrombin-containing liquid; adding a chelator to complex calcium in the thrombin-containing liquid; adjusting the pH of the thrombin-containing liquid to below 7; and reducing the temperature of the thrombin-containing liquid.

23. The method of claim 22, wherein the alcohol is glycerol.

24. The method of claim 12, wherein the blood component comprising platelets is obtained from the subject.

25. The method of claim 12, wherein applying the thrombin-containing liquid to the site on the subject to form a clot comprises combining the thrombin-containing liquid with fibrinogen.
